SEQUENCE LISTING (4 sequences)

SEQ ID NO: 1 - protein, 5 aa, Artificial Sequence
  Synthetic
LXXLL

SEQ ID NO: 2 - DNA, 20 bp, Artificial Sequence
  Oligonucleotide designed to act as RT-PCR primer.
tgatttgggggattgctata

SEQ ID NO: 3 - DNA, 19 bp, Artificial Sequence
  Oligonucleotide designed to act as RT-PCR primer.
catacctgggctgtgctct

SEQ ID NO: 4 - DNA, 23 bp, Artificial Sequence
  Oligonucleotide designed to act as RT-PCR primer.
tggttcaccgtttgccttctcct

(19) United States
(12) Patent Application Publication
(10) Pub. No.: US 2002/0119958 A1
(43) Pub. Date: Aug. 29, 2002

(54) Therapeutic agent for hyperlipidemia

(76) Inventors: Shinichiro Tojo, Ashiya-shi (JP); Masahiro Nita, Sakai-shi (JP); Takeshi Nishimura, Huddinge (SE); Bei Shan, Redwood City, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 09/782,535

(22) Filed: Feb. 13, 2001

(51) Int. Cl. A61K 31/165; A61K 31/56

(52) U.S. Cl. 514/178; 514/617

(57) ABSTRACT

The present invention provides a therapeutic agent for hyperlipidemia, which has a novel action mechanism and which contains a farnesoid X receptor (FXR) antagonist as an active ingredient, and a screening method of the antagonist.

### TECHNICAL FIELD OF THE INVENTION

The present invention relates to a therapeutic agent for hyperlipidemia, which contains a farnesoid X receptor (FXR) antagonist as an active ingredient.

### BACKGROUND OF THE INVENTION

The conversion of cholesterol into bile acid in the liver starts with hydroxylation at the 7-position of cholesterol. Synthesized bile acid is discharged from the liver into the intestine, and promotes intestinal absorption of fat derived from meals. The bile acid in the intestine is mostly re-absorbed actively and efficiently in the ileum, and returns to the liver through portal.

The hydroxylation of the 7-position of cholesterol, which is a first step and a rate-determining step for the synthesis of bile acid, is catalyzed by cholesterol 7α-hydroxylase (CYP7A) that expresses liver-specifically. The expression level of CYP7A gene encoding CYP7A is subject to feedback inhibition by the final product, bile acid, as a result of which the expression is repressed [Vlahcevic, Z. R. et al., Hepatology 13, 590-600 (1991)].

Release of the repression of CYP7A gene expression by bile acid and constant promotion of the expression of CYP7A gene is expected to decrease cholesterol in the liver and subsequently promote expression of LDL receptor gene in the liver, thereby leading to a lower serum cholesterol value.

In fact, a report has documented that a forcible expression of CYP7A gene using an adenovirus vector in the liver of hamster resulted in a lower serum cholesterol value, which supports this idea [Spady, D. K. et al., J. Clin. Invest. 96, 700-709 (1995)].

Nuclear receptor is a transcription factor which is activated by the binding of a ligand and controls the expression of a target gene, and thus plays an important role in various physiological phenomena. In 1999, a report was published stating that the ligand of FXR (one of the nuclear receptors) is a bile acid molecule exemplified by chenodeoxycholic acid (CDCA), and that the transcription activity of FXR is potentiated by CDCA [Makishima, M. et al. Science 284, 1362-5 (1999), Parks, D. J. et al., Science 284, 1365-8 (1999), Wang, H. et al., Mol. Cell 3, 543-53 (1999)].

When a ligand is not bound, a corepressor is bound with a nuclear receptor. This corepressor is considered to deacetylate histone to make a chromatin structure dense and repressively act on the initiation of transcription. When a ligand is bound with a nuclear receptor, the ligand binding domain of the receptor comes to have a different helical structure, and the different structure enables binding of a coactivator (forms a complex with a different protein to be operable) with the receptor. With the binding of a coactivator, a repressor is released and the transcription is derepressed. The coactivator acetylates histone, thereby actively changing the chromatin structure, which in turn affords smooth initiation of transcription, thus exhibiting a transcription-promoting action. As shown above, a nuclear receptor is strictly ligand dependent.

In the liver, the expression of CYP7A gene is subject to feedback repression by CDCA. The CDCA-dependent repression of transcription has been shown to be indirectly controllable by FXR, for which CDCA is a ligand. In other words, FXR CDCA-dependently activates the transcription of a transcription factor SHP (small heterodimer partner) gene that negatively controls the expression of CYP7A gene, and indirectly represses expression of CYP7A gene [Lu T T et al., Mol. Cell. 6, 507-15 (2000), Goodwin B et al., Mol. Cell. 6, 517-26 (2000)].

On the other hand, FXR has been shown to promote expression of an intestinal bile acid-binding protein (I-BABP) gene [Makishima, M. et al. Science 284, 1362-5 (1999)]. I-BABP is a cytoplasmic protein that specifically expresses in ileum epitheliocytes and has been shown to bind with bile acid [Kramer, W. et al., J. Biol. Chem., 268, 18035-46 (1993)]. I-BABP, from its expression site and the binding capacity with bile acid, is a molecule postulated to be involved in the active intestinal re-absorption of bile acid in the ileum. When the gene expression of I-BABP is repressed, re-absorption of bile acid in the ileum is repressed, which in turn may reduce the amount of bile acid that returns to the liver, and therefore, promote the expression of CYP7A gene. From this aspect, an FXR antagonist has a potential of inducing repression of I-BABP gene expression in the ileum, leading to a reduced level of serum cholesterol.

In view of these actions, there is a possibility that an FXR antagonist promotes synthesis of bile acid by derepression of the expression of CYP7A gene, and the repressed expression of I-BABP gene results in the repression of re-absorption of bile acid from the intestine, which in turn reduces the amount of bile acid that returns to the liver, whereby the serum cholesterol level is lowered.

When Sinal et al. prepared an FXR gene knockout mouse and examined the phenotype [Sinal, C. J. et al., Cell 102, 731-44 (2000)], however, mice without FXR gene showed increased levels of cholesterol and neutral fats in the liver, as well as increased levels of serum cholesterol and neutral fats, as compared to wild type mice. This report denies the possibility of a serum lipid reducing agent that is based on the antagonism to the FXR function. Thus, a suggestion with regard to a definite relationship between the FXR function and the metabolism of fat, particularly between the cholesterol metabolism and transport of bile acid, has been awaited.

As a factor involved in the transport of bile acid in the liver, a bile salt export pump (Bsep) is known. Bsep is a primary molecule responsible for the excretion of bile acid into the bile duct in the liver of mammals [Gerloff, T. et al., J. Biol. Chem., 273, 10046-10020 (1998)]. Functional inhibition or lower amount of expression of Bsep gene is considered to cause lower excretion of bile acid and bile stasis in the liver. Lower amounts of Bsep gene expression are observed in FXR defective mice, which suggests the possibility of FXR positively controlling the expression of Bsep gene. However, the phenotype observed in the FXR defective mice may be a secondary effect derived from deletion of FXR during embryogenesis, and there is no report on the influence of FXR agonist and antagonist on the Bsep gene transcription.

### SUMMARY OF THE INVENTION

An object of the present invention is to provide a therapeutic agent for hyperlipidemia, which has a new action mechanism, and a method for screening a farnesoid X receptor (FXR) antagonist which is an active ingredient of a therapeutic agent for hyperlipidemia.

The present inventors have made intensive studies in view of the above-mentioned problems, and now found that an FXR antagonist increases the expression of a CYP7A gene or protein and represses the expression of an I-BABP gene or protein, and established that this antagonist is useful as a therapeutic agent for hyperlipidemia, which resulted in the completion of the present invention. They have further found that a compound having an FXR ligand-dependent inhibitory action, particularly a compound that increases excretion of bile acid in a liver-derived cell line, is useful as a therapeutic agent for hyperlipidemia. In addition, they have succeeded in constructing a novel method for screening an FXR antagonist.

Accordingly, the present invention provides the following.

[1] A method for treating hyperlipidemia, which comprises administering a pharmaceutically effective amount of a farnesoid X receptor (FXR) antagonist to a patient.

[2] The method of the above-mentioned [1], wherein the FXR antagonist has an IC₅₀ value of not more than 10 μM.

[3] The method of the above-mentioned [1], wherein the IC₅₀ value of the FXR antagonist is not more than 10 μM when the ligand is 100 μM.

[4] The method according to the above-mentioned [3], wherein the FXR ligand is a bile acid.

[5] The method according to the above-mentioned [4], wherein the bile acid is chenodeoxycholic acid, deoxycholic acid, lithocholic acid, ursodeoxycholic acid or 3,7-diketocholanic acid.

[6] The method according to the above-mentioned [1], wherein the FXR antagonist is N-(3,5-di-tert-butyl-2,6-dihydroxyphenyl)benzamide or a pharmaceutically acceptable salt thereof.

[7] A method for treating hyperlipidemia, which comprises repressing a ligand dependent action of FXR.

[8] A method for promoting biosynthesis of bile acid, which comprises increasing the expression of a cholesterol 7α-hydroxylase (CYP7A) gene or protein.

[9] A method for inhibiting re-absorption of bile acid, which comprises repressing the expression of an intestinal bile acid-binding protein (I-BABP) gene or protein.

[10] A method for promoting bile acid secretion, which comprises prohibiting decrease of expression of a bile salt export pump (Bsep) gene or protein.

[11] The method according to the above-mentioned [1] or [7], which shows at least one of the following features (a) to (c):

(a) increase in the expression of a CYP7A gene or protein

(b) repression of the expression of an I-BABP gene or protein

(c) prohibition of a decrease in the expression of a Bsep gene or protein.

[12] A method for screening an FXR antagonist, which comprises the following steps:

(1) forming, in the presence of bile acid, a complex of FXR or its operable fragment labeled with a first fluorescent dye and an FXR coactivator labeled with a second fluorescent dye,

(2) adding a test compound and incubating the compound, and

(3) measuring an amount of a free coactivator by a fluorescence resonance energy transfer assay method.

[13] The screening method according to the above-mentioned [12], wherein the bile acid is chenodeoxycholic acid.

[14] The screening method according to the above-mentioned [12], wherein the FXR coactivator is that selected from the SRC-1 family or its operable fragment.

[15] An FXR antagonist obtained by the screening method of any of the above-mentioned [12] to [14].

[16] A method for treating hyperlipidemia, which comprises administering a pharmaceutically effective amount of an FXR antagonist to a patient.

### BRIEF DESCRIPTION OF THE DRAWINGS

FIG. 1 is a graph showing an inhibitory effect by compound A [N-(3,5-di-tert-butyl-2,6-dihydroxyphenyl)benzamide] on the CDCA-induced binding of an FXR ligand binding domain and a coactivator SRC-1, and the specificity thereof, wherein the vertical axis shows fluorescent intensity and the horizontal axis shows the concentration of compound A.

FIG. 2 is a graph showing an inhibitory effect by compound A on the CDCA-induced transcription activity of a reporter gene via FXR and the specificity thereof, wherein the vertical axis shows the activation level of the reporter gene, in which, in the absence of a drug, the value upon activation by ligand in the absence of a drug was 100% and the value in the absence of a ligand was 0%, and the horizontal axis shows the concentration of compound A.

FIG. 3 shows an inhibitory effect by compound A on the repression of CYP7A expression by CDCA in HepG2 cells, as examined by western blot analysis, wherein the concentration of CDCA was maintained constant (20 μM), and the concentration of compound A was changed to various values.

FIG. 4 shows an inhibitory effect by compound A on the potentiating action on I-BABP mRNA expression by CDCA in Caco-2 cells, as examined by northern blot analysis, wherein the concentration of CDCA was maintained constant (100 μM) and the concentration of compound A was changed to various values.

FIG. 5 shows a promoting effect by compound A on excretion of bile acid in HepG2 cells, wherein the vertical axis shows the amount of excreted bile acid per the protein amount of the cell, based on the value without addition of the compound as 100%.

FIG. 6 is a graph showing changes of the concentration of compound A in blood when the compound was forcibly administered once orally to hamsters, wherein the vertical axis shows the concentration of compound A in blood and the horizontal axis shows time (min) after oral administration.

FIG. 7 is a graph showing an effect by compound A on hamster serum cholesterol, wherein the vertical axis shows the total cholesterol concentration in the serum and the horizontal axis shows time (days) after oral administration.

FIG. 8A is a graph showing an influence of the administration of compound A on the expression of CYP7A gene in the liver of hamster as examined by northern blot analysis, and

FIG. 8B is a graph showing the influence of the administration of compound A on the expression of I-BABP gene in the ileum of hamster as examined by northern blot analysis.

### DETAILED DESCRIPTION OF THE INVENTION

In the present invention, by the “farnesoid X receptor (FXR) antagonist” is meant a substance that inhibits ligand-dependent induction of transcription caused by FXR. More specifically, it is a substance that competitively inhibits the binding of FXR and a coactivator of the receptor in the presence of a ligand. Such FXR antagonist can be obtained by the screening method of the present invention, which is to be mentioned later. For example, a compound having the following structure and a pharmaceutically acceptable salt thereof are exemplified.

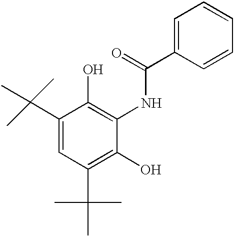

In the present invention, a “pharmaceutically acceptable salt” may be any as long as it forms a non-toxic salt with this compound. For example, there are mentioned salt with inorganic acid such as hydrochloride, hydrobromate, hydroiodate, sulfate, nitrate, phosphate, carbonate, hydrogencarbonate, perchlorate and the like; salt with organic acid such as formate, acetate, trifluoroacetate, propionate, oxalate, glycolate, succinate, lactate, maleate, hydroxymaleate, methylmaleate, fumarate, adipate, tartrate, malate, citrate, benzoate, cinnamate, ascorbate, salicylate, 2-acetoxybenzoate, nicotinate, isonicotinate and the like; sulfonate such as methanesulfonate, ethanesulfonate, isethionate, benzenesulfonate, p-toluenesulfonate, naphthalenesulfonate and the like; salt with acidic amino acid such as aspartate, glutamate and the like; alkali metal salt such as sodium salt, potassium salt and the like; alkaline earth metal salt such as magnesium salt, calcium salt and the like; ammonium salt; salt with organic base such as trimethylamine salt, triethylamine salt, pyridine salt, picoline salt, dicyclohexylamine salt, N,N′-dibenzylethylenediamine salt and the like; amino acid salt such as lysine salt, arginine salt and the like; and the like.

Preferably, this antagonist shows an antagonistic action as evidenced by IC₅₀ of not more than 10 μM, more preferably not more than 1 μM, when the ligand concentration is 100 μM.

The ligand is not subject to any particular limitation as long as FXR after binding with the ligand can promote transcription, and may be a known substance. Specifically, it is bile acid, preferably chenodeoxycholic acid, deoxycholic acid, lithocholic acid, ursodeoxycholic acid, 3,7-diketocholanic acid and the like, more preferably chenodeoxycholic acid having a particularly strong FXR activation capability.

This antagonist preferably shows at least one of, more preferably all of, the following characteristics:

(a) increasing the expression of a CYP7A gene or protein

(b) repressing the expression of an I-BABP gene or protein

(c) prohibiting decrease of expression of a Bsep gene or protein.

An FXR antagonist (when a salt exists, inclusive of such salt) can be used in the form of a solid, semisolid or liquid in admixture with an organic or inorganic carrier or excipient suitable for oral or parenteral application. The antagonist can be admixed with a conventional, non-toxic and pharmaceutically acceptable carrier for a dosage form suitable for use, such as powder, tablet, pellet, capsule, suppository, liquid, emulsion, suspension, aerosol, spray and the like. Where necessary, auxiliaries, stabilizer, thickener and the like can be used. These carriers and excipients may undergo a sterilization treatment as necessary, or may be subjected to a sterilization treatment after producing a preparation.

The amount of FXR antagonist (active ingredient) effective for the treatment varies depending on the age, condition and the like of individual patients to be treated and is determined depending on these factors. The FXR antagonist has an antagonistic action against FXR in mammals, such as human, cow, horse, dog, mouse, rat and the like. The antagonistic action is a CYP7A gene or protein expression increasing action and an I-BABP gene or protein expression repressing action, and preferably in addition to these ligand dependent actions, an action to prevent decrease of the expression of a Bsep gene or protein. Thus, the FXR antagonist is useful as a medicament for the prophylaxis and treatment of diseases relating to cholesterol metabolism and bile acid transport, particularly for the prophylaxis and treatment of hyperlipidemia showing increase in serum cholesterol as a main symptom.

The FXR antagonist usable in the present invention may be that conveniently obtained by the screening method (to be mentioned later) of the FXR antagonist of the present invention. It also encompasses a compound known or expected to have the antagonistic action.

The inventive method for screening the FXR antagonist is explained in detail according to each step.

Step 1:

A step for forming a complex of an FXR or its operable fragment labeled with a first fluorescent dye and an FXR coactivator labeled with a second fluorescent dye, in the presence of bile acid.

The bile acid to be used in this step is exemplified by those mentioned above, which is preferably chenodeoxycholic acid.

The FXR to be used in the present invention may be naturally derived or may be obtained by genetic engineering such as gene recombination and the like. It is also possible to synthesize or semisynthesize based on a known amino acid sequence. Moreover, the preparation, isolation and purification thereof may be performed by combining known methods. As long as the ligand binding capacity and ligand dependent transcription induction can be achieved, the FXR to be used for the screening method of the present invention may have one or more substituted, deleted or added amino acids in the amino acid sequence, and such protein is also encompassed in the FXR of the present invention.

According to the present invention, moreover, an operable fragment of the above-mentioned FXR can be also used in a similar manner. By the “operable fragment of FXR” is meant one having a capability to bind with a ligand and a coactivator, as well as to induce ligand-dependent transcription, which intends a polypeptide or a protein having a partial amino acid sequence of the above-mentioned FXR. For example, there is mentioned a polypeptide or a protein containing a region called a ligand binding domain (LBD) (Broaowski, A. M. et al., Nature. 389, 753-758 (1997)).

The first and second fluorescent dyes used as labeling dye in this step are a pair of fluorescent dye molecules capable of causing Fluorescence Resonance Energy Transfer: FRET. In FRET, one of these fluorescent dye molecules is a donor fluorescent dye molecule of energy and the other is a receptor thereof (acceptor fluorescent dye molecule). FRET refers to a phenomenon where certain two fluorescent compounds are located near (approximately within a distance of 100 Å) and the fluorescent spectrum of one (donor fluorescent dye molecule) of the two fluorescent compounds and the excitation spectrum of the other (acceptor fluorescent dye molecule) overlap with each other, and when energy at the excitation wavelength of the donor is applied, the fluorescence of the donor, which should be observed under normal circumstances, is attenuated, and instead, the fluorescence of the acceptor is observed. For example, when a coactivator and FXR form a complex, only the fluorescence of the acceptor is observed and when the coactivator is liberated due to the antagonist, the fluorescence of the donor is observed.

Specifically, the donor fluorescent dye molecule is exemplified by fluorescein, fluorescein isothiocyanate (FITC), allophycocyanin (APC) and the like, and the acceptor fluorescent dye molecule is exemplified by x-Rhodamine, Tetramethylrhodamine isothiocyanate (TRITC), carbocyanine 3 (CY3), euflavine (Eu) and the like. These fluorescent dyes are commercially available. Using such fluorescent dyes, FXR or a coactivator can be fluorescent labeled by a conventional method. That is, a fluorescent-labeled FXR is obtained by preparing a fusion protein of FXR and GST (glutathione S-transferase) and mixing it with a fluorescent dye-conjugated anti-GST antibody. A fluorescent-labeled coactivator is obtained by biotinating a coactivator and mixing it with a fluorescent dye-conjugated streptavidin (Zhou, G. et al., Mol. Endocrinol., 12, 1594-1604 (1998), Makishima, M. et al., Science, 284, 1362-1365 (1999)).

The FXR coactivator is not subject to any particular limitation as long as it can bind with FXR in a ligand dependent manner and various known coactivators can be used. A preferable coactivator makes, upon binding, FXR promote transcription. Such coactivator may be, for example, a protein belonging to an SRC-1 family, which is more specifically exemplified by a series of proteins having high homology, such as SRC-1, TIF2, AIBI and the like, preferably SRC-1. Where necessary, a different protein (e.g., CBP) may be bound. The protein may have an amino acid sequence wherein one or more amino acids are substituted, deleted or added, as long as it can bind with FXR in a ligand dependent manner. Such protein is also encompassed in the FXR coactivator of the present invention. As mentioned earlier, the antagonist of the present invention inhibits induction of transcription by FXR as a result of competition with the coactivator.

As used herein, by the “ligand dependent” is meant the need of a ligand for the binding of a coactivator and FXR, and such ligand forms a complex with the FXR and coactivator.

According to the present invention, moreover, an operable fragment of a coactivator can be used in addition to the above-mentioned coactivator. By the “operable fragment of coactivator” is meant a fragment that binds with FXR in the presence of a ligand, preferably such fragment additionally having a function of a coactivator. It is, for example, a polypeptide or protein having a partial amino acid sequence of the above-mentioned coactivator, which is more specifically a polypeptide or protein containing an LXXLL (SEQ ID NO:1) motif. Such motif is known as a nuclear receptor binding sequence (Herry, D. M. et al., Nature 387, 733-736 (1997)).

The reaction conditions of Step 1 are appropriately determined according to bile acid to be used as a ligand, a fluorescent dye to be used as a label, the kind of the coactivator and the like.

Step 2:

A step for addition of a test compound and incubation of the compound.

As used herein, by the “test compound” is meant a compound selected or synthesized for the purpose of examining the presence or otherwise of an FXR antagonistic action, and the term encompasses novel compounds and known compounds reported to have different actions. The test compound is added in an amount determined according to the kind of the compound, and preferably tested for antagonistic activity by serially changing the amount thereof.

Generally, the above-mentioned Step 1 and Step 2 are conducted simultaneously. To be specific, in the presence of a ligand, a labeled coactivator having an approximately 10-fold concentration is added to a labeled FXR and incubated at a constant temperature (preferably about 4° C.) for several hours to overnight (preferably about 12 h). As the reaction solution, various buffers generally used in this field are used, such as Hepes buffer.

Step 3:

A step for measurement of an amount of free coactivator by FRET assay method.

The coactivator is released from the complex of FXR and the coactivator formed in Step 1, when the test compound shows an antagonistic action after incubation in Step 2. The release of the coactivator obliterates the FRET phenomenon. Changes in fluorescence resulting therefrom are measured using a fluorescence photometer and the like.

For example, when the combination of the first fluorescent dye and the second fluorescent dye is that of Eu and APC, they are excited at 337 nm and measured for fluorescence (620 nm for Eu and 665 nm for APC), based on which the fluorescence ratio of 665 nm/620 nm is taken as an FRET fluorescence intensity (Makishima, M. et al., Science, 284, 1362-1365 (1999)). The fluorescence can be measured using an apparatus such as Victor II olate Reader (Wallac) and the like.

The FXR antagonist obtained by the screening method of the present invention can be used for, besides the use as a therapeutic agent for hyperlipidemia mentioned above, various applications wherein its action is useful. For example, it can be a useful tool for the analysis of the action mechanism of the liposoluble signal molecule via a nuclear receptor, particularly FXR, and for the study of various diseases which are closely related to the cholesterol metabolism and the transport of bile acid, such as hyperlipidemia.

### EXAMPLES

The present invention is specifically explained in detail in the following by referring to Examples, by which the present invention is not limited. In the examples, a compound of the following formula (hereinafter to be conveniently referred to as compound A) was used as a test compound:

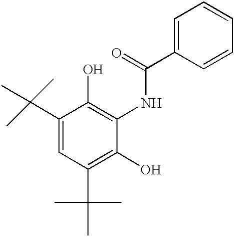

The compound A can be synthesized as in the following Preparation Example.

### Preparation Example

a) Synthesis of 2-aminoresorcinol

A mixture of 2-nitroresorcinol (30.2 g, 0.195 mmol) and 10% Pd-C (315 mg) in methanol (500 mL) was stirred at room temperature for 2 days under a hydrogen atmosphere. The reaction mixture was filtered through Celite, washed with methanol, and the obtained filtrate was concentrated under reduced pressure. The resulting residue was purified by silica gel column chromatography (methanol:dichloromethane=1:9) to give a crude fraction (24 g) of the objective 2-aminoresorcinol, which was used in the next reaction without further purification.

b) Synthesis of N-(2,6-dihydroxyphenyl)benzamide

To a solution (400 mL) of 2-aminoresorcinol (24 g) and triethylamine (135 mL, 0.969 mol) in tetrahydrofuran (THF) was added dropwise benzoyl chloride (23.0 mL, 0.198 mol) at 0° C. under a nitrogen atmosphere. The mixture was stirred overnight at room temperature, and 5 N aqueous potassium hydroxide solution (200 mL) was added, which was followed by stirring for 2 more hours at room temperature. The reaction mixture was adjusted to pH 2 and extracted with ethyl acetate. The organic layer was washed with brine, dried over magnesium sulfate, filtrated, and concentrated under reduced pressure. The obtained residue was purified twice by silica gel column chromatography (first time; dichloromethane:ethyl acetate=20:1→10:1, second time; hexane:ethyl acetate=4:1→1:1) to give the objective N-(2,6-dihydroxyphenyl)benzamide (30.3 g, yield from 2-nitroresorcinol 68%).

c) Synthesis of N-(3,5-di-tert-butyl-2,6-dihydroxyphenyl)benzamide

To a suspension of N-(2,6-dihydroxyphenyl)benzamide (15.0 g, 65.4 mmol) in phosphoric acid (300 mL) was added tert-butyl alcohol (190 mL, 1.96 mol) gently at room temperature under a nitrogen atmosphere, and the mixture was stirred at 50° C. overnight. The reaction mixture was cooled, diluted with water and extracted with dichloromethane and ethyl acetate. The both organic layers were combined, washed with saturated brine, dried over magnesium sulfate, filtrated, and concentrated under reduced pressure. The obtained residue was purified by silica gel column chromatography (hexane:ethyl acetate=9:1→4:1) to give the objective N-(3,5-di-tert-butyl-2,6-dihydroxyphenyl)benzamide (8.96 g, yield 40%).

### Example 1

Inhibitory Effect of Compound A on CDCA-Induced Binding of FXR Ligand Binding Domain to Coactivator SRC-1, and Specificity of the Inhibition

An Eu-labeled FXR ligand binding domain (FXR-LBD) and an APC-labeled coactivator SRC-1 were mixed to the final concentration of 10 nM and 100 nM, respectively, in a buffer [100 mM Hepes (pH 7.6), 0.125% CHAPS, 125 mM NaF] in the presence of ligand CDCA. Thereto were added various concentrations of compound A (1-50 μM), and the mixture was allowed to react at 4° C. for 12 h. After the reaction, it was exposed to an excitation light at 337 nm and the fluorescence at 665 nm and 620 nm was measured, based on which the fluorescence ratio of 665 nm/620 nm was taken as an FRET fluorescence intensity.

The results are shown in FIG. 1. At a concentration of not less than 1 μM, compound A inhibited the binding between FXR-LBD and SRC-1 induced in the presence of CDCA (attenuation of FRET fluorescent intensity). For a test for comparison, a similar experiment was conducted using LXR (liver X receptor) ligand binding domain (LXR-LBD) instead of FXR-LBD. The compound A did not affect the fluorescent intensity up to 50 μM.

The results reveal that compound A has a specific FXR antagonistic action.

### Example 2

Inhibitory Effect of Compound A on CDCA-Induced Transcriptional Activation of Reporter Gene Via FXR, and Specificity Thereof

A plasmid for compulsory expression of FXR, a luciferase reporter plasmid and a β-galactosidase expression plasmid for correction of gene transfer were introduced into cultured mammalian cells (293 or CV-1) by conventional methods. The cells after gene transfer were treated with CDCA and compound A. Cell lysates were prepared from the cells that underwent treatment with various concentrations of compound A (0.1-10 μM), and a luciferase activity (corrected based on β-galactosidase activity) in the cell lysate was expressed as an FXR dependent transcriptional activity. In the absence of a drug, the luciferase activity value upon activation with CDCA was taken as 100% and the value in the absence of a ligand was taken as 0%. For a test for comparison, a similar experiment was conducted using an LXR expression plasmid instead of the FXR expression plasmid.

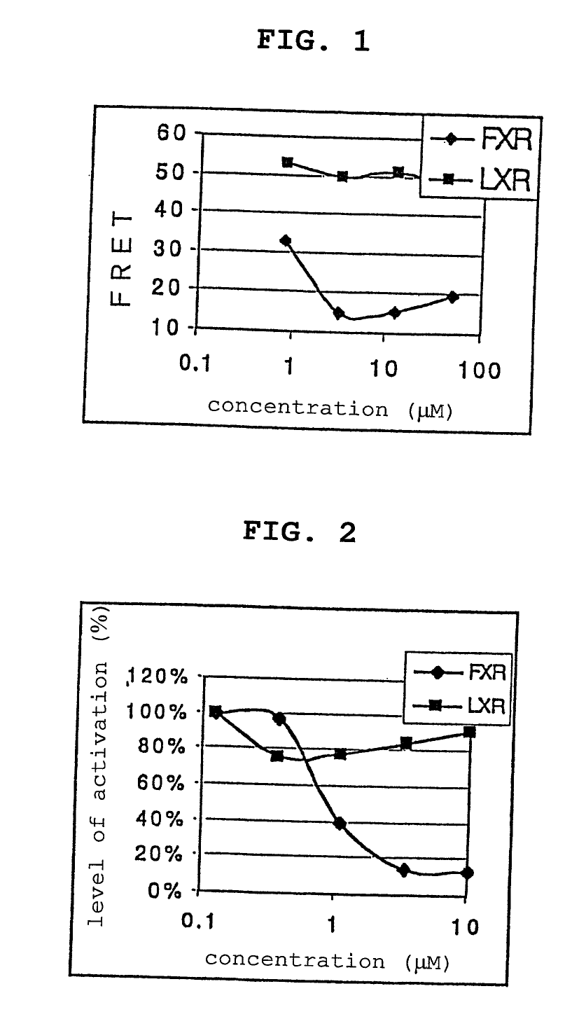

The results are shown in FIG. 2. By the addition of compound A, an FXR dependent transcription was repressed at a concentration of not less than 1 μM.

In contrast, compound A did not influence the transcription activity dependent on LXR, which is the other nuclear receptor, up to the concentration of 10 μM. From these results, it was considered that the transcription repressing activity of compound A was FXR specific.

### Example 3

Inhibitory Effect of Compound A on CDCA-Induced Repression of CYP7A Expression in HepG2 Cells

The CDCA, ligand of FXR, is reported to repress the expression of CYP7A gene in human hepatoma-derived cell line, HepG2, at an mRNA level and a protein level (Makishima, M. et al. Science 284, 1362-5 (1999)).

The effect of compound A, which is an FXR antagonist, on the expression of CYP7A gene was tested using HepG2 cells.

(1) Analysis at mRNA Level

The cultured HepG2 cells were treated under the following three conditions at 37° C. for 16 h.

1) no treatment with CDCA, no treatment with compound A

2) treatment with CDCA (20 μM), no treatment with compound A

3) treatment with CDCA (20 μM), treatment with compound A (10 μM)

RNA was prepared from the cells after the treatment and the amount of CYP7A mRNA contained in the RNA was measured by quantitative RT-PCR. For the quantitative RT-PCR, TaqMan One Step Gold reverse transcriptase PCR kit of Applied Biosystems/PerkinElmer was used. For quantification, the following primers (i)-(iii) derived from human CYP7A gene sequence were used.

(i) 5′-TGATTTGGGGGATTGCTATA (SEQ ID NO: 2)

(ii) 5′-CATACCTGGGCTGTGCTCT (SEQ ID NO: 3)

(iii) 5′-TGGTTCACCGTTTGCCTTCTCCT (SEQ ID NO: 4) labeled by the use of 6-FAM(6-carboxyfluorescein) (5′-end) and TAMRA (n,n,n′,n′-tetramethyl-6-carboxyrhodamine) (3′-end)

When the amount of CYP7A mRNA without a treatment with CDCA (condition 1) was 100 and the amount of CYP7A mRNA treated with 20 μM CDCA (condition 2)) was 0, the addition of compound A under the CDCA treatment conditions (condition 3) made the amount of CYP7A mRNA 58.

(2) Analysis at Protein Level

The cultured HepG2 cells were treated under the following three conditions at 37° C. for 16 h.

1) no treatment with CDCA, no treatment with compound A

2) treatment with CDCA (20 μM), no treatment with compound A

3) treatment with CDCA (20 μM), treatment with compound A (0.03, 0.1, 0.3, 1, 3, 10 μM)

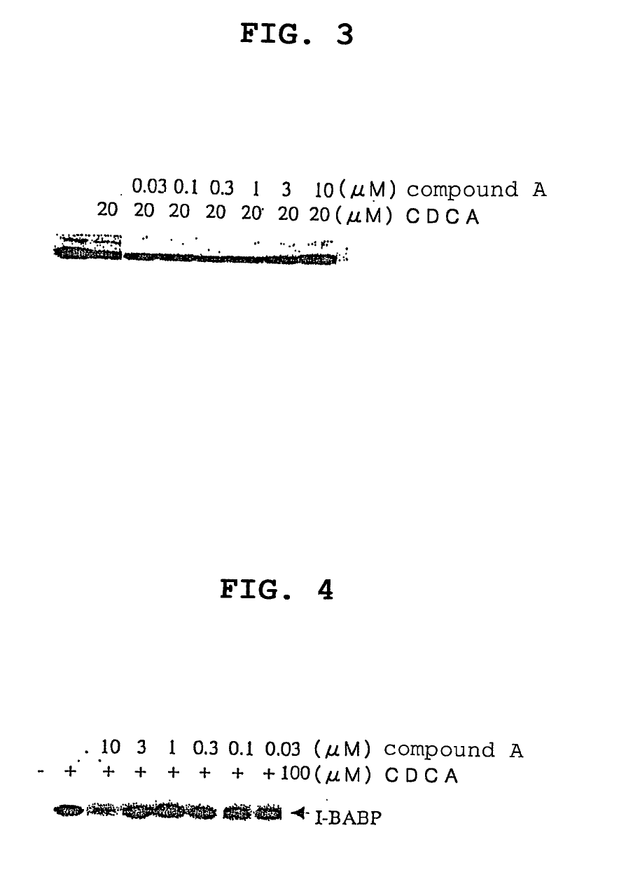

After the treatment, the cells were recovered and lysed in a lysis buffer (125 mM Tris HCl (pH 8.0), 2 mM CaCl₂, 2% Triton X-100) to prepare a cell lysate. The cell lysate was fractionated by SDS-PAGE, and the protein was transferred onto a PVDF membrane. The CYP7A protein was detected by Western blot analysis using an anti CYP7A antibody and an HRP (horse-radish peroxidase) conjugated 2nd antibody. The results are shown in FIG. 3. Accumulation of CYP7A protein by compound A was observed.

### Example 4

Inhibitory Effect of Compound A on CDCA-Induced Potentiation of I-BABP Expression in Caco-2 Cells

The human colon carcinoma derived cell line, Caco-2, is a cell line differentiated like the small intestine and expresses the I-BABP gene. The I-BABP gene expression in Caco-2 cells is promoted by CDCA and an expression control system of I-BABP gene by FXR is considered to be also present in Caco-2 cells (Makishima, M. et al. Science 284, 1362-5 (1999)). Thus, an action on the I-BABP gene expression of compound A as an FXR antagonist was tested using Caco-2 cells.

The Caco-2 cells were cultured in the presence of 100 μM CDCA, and the expression of I-BABP gene was induced. Thereto was added compound A and changes in the I-BABP gene expression level were examined by measuring the amount of I-BABP mRNA by Northern blot analysis.

The results are shown in FIG. 4. The compound A caused a marked decrease in the mRNA level of I-BABP at 10 μM in Caco-2 cells.

### Example 5

Influence of Compound A on Excretion of Bile Acid from HepG2 Cells

The cultured HepG2 cells were plated in a 24-well culture dish at 1.5×10⁵ cells/ml, 0.5 ml/well. Four days later, the medium was exchanged to a new one and ¹⁴C-labeled cholesterol (18.5 kBq/well) was added, which was cultured for 24 h (bile acid in the cells was labeled thereby). After the culture, the medium was exchanged to a new one and compound A (final concentration 1, 10 μM) was added. After 24 hours of culture, the medium was recovered and the amount of bile acid excreted into the medium was measured to examine the influence of compound A on bile acid excretion. The amount of excreted bile acid was corrected based on the protein amount of the cell. The results are shown in FIG. 5. The compound A promoted the excretion of bile acid from HepG2 cells.

In view of the result that compound A as an FXR antagonist has a bile acid excretion-promoting action, it is less likely that FXR positively controls the Bsep gene expression. Such speculation is clarified by examining the influence on Bsep gene expression using this antagonist.

### Example 6

The cultured HepG2 cells are plated in a 6-well culture dish at 3×10⁵ cells/ml, 2 ml/well, and compound A (final conc. 0.1-10 μM) is added the next day. After culture for 4-24 h, the total RNA is prepared by AGPC (acid guanidinium phenol chloroform) method. The prepared RNA is separated by agarose electrophoresis and transferred onto a nylon membrane. A human Bsep gene is cloned from human liver derived RNA by RT-PCR, and using this, ³²P-labeled probe is prepared. The prepared probe is hybridized with the nylon membrane after RNA transfer in 50% formamide at 42° C. for 16 h, and influence of compound A on Bsep gene expression in the human liver derived cell line is examined.

### Example 7

The compound A is orally administered (30 or 100 mg/kg/day) to 7-week-old male Syrian Golden Hamster. After oral administration for 7 to 14 days, the liver is removed from the hamster. The total RNA is prepared from the obtained liver by AGPC method. The prepared RNA is separated by agarose electrophoresis and transferred onto a nylon membrane. A hamster Bsep gene is cloned from RNA derived from the liver of the hamster by RT-PCR, and using this, ³²P-labeled probe is prepared. The prepared probe is hybridized with the nylon membrane after RNA transfer in 50% formamide at 42° C. for 16 h, and influence of compound A on Bsep gene expression in the hamster liver is examined.

### Example 8

Transition of Compound A After its Oral Administration to Hamster, in Blood

The compound A was forcibly given to hamster by single oral administration and measured for shifts in the concentration in blood. The test conditions were as follows.

(1) Animal Test Conditions

animal: male GS hamster (purchased from Japan SLC), 9 weeks old administration liquid: 20 mg/ml 0.5% methylcellulose suspension diet: not fasted

dose: 100 mg/5 ml/kg (p.o.)

administration: forcible single oral administration using oral sound

blood sampling time: 5, 15, 30, 60, 180, 360 min after administration

serum preparation: blood was drawn from the orbital vein and

centrifuged using a Separapid tube (coagulation promoting spitz tube containing serum separating agent) to give serum.

(2) Analysis of Compound A

An internal standard substance (CV-5386, 0.1 μg) was added to serum (0.1 ml) and admixed. Methanol (0.3 ml) was added and the mixture was stirred. The mixture was centrifuged at 10,000 rpm (FORCE-7 Denver Instrument Company) for 2 min and the resulting supernatant was subjected to centrifugal filtration using Centricut. The obtained filtrate was analyzed by LC-ESI-MS/MS.

(3) Results

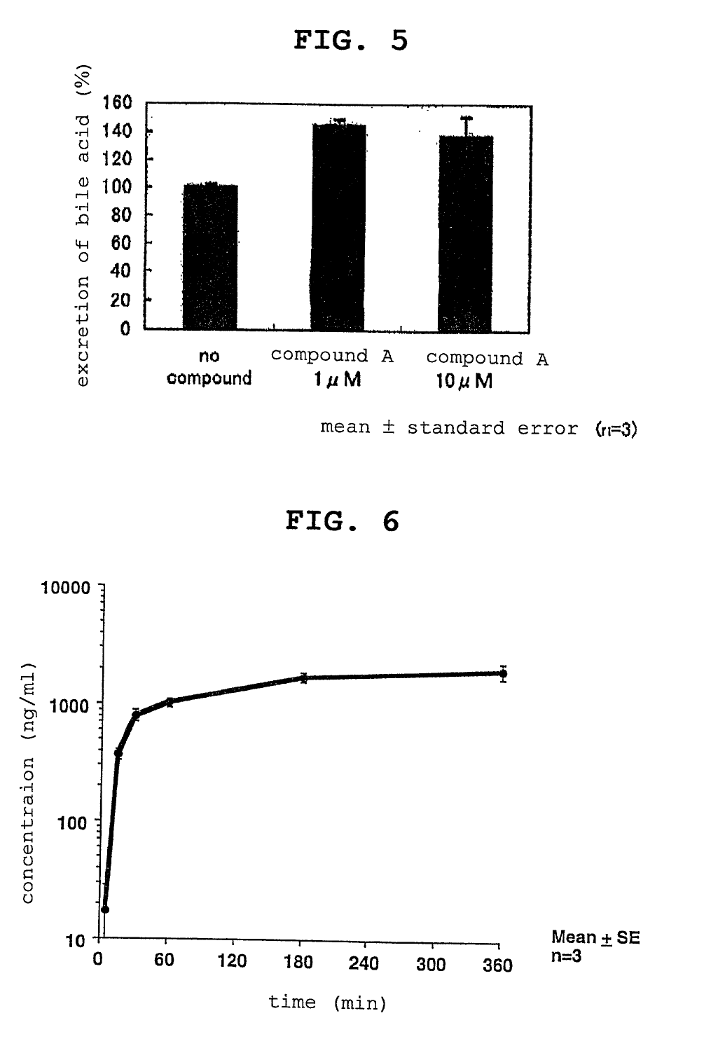

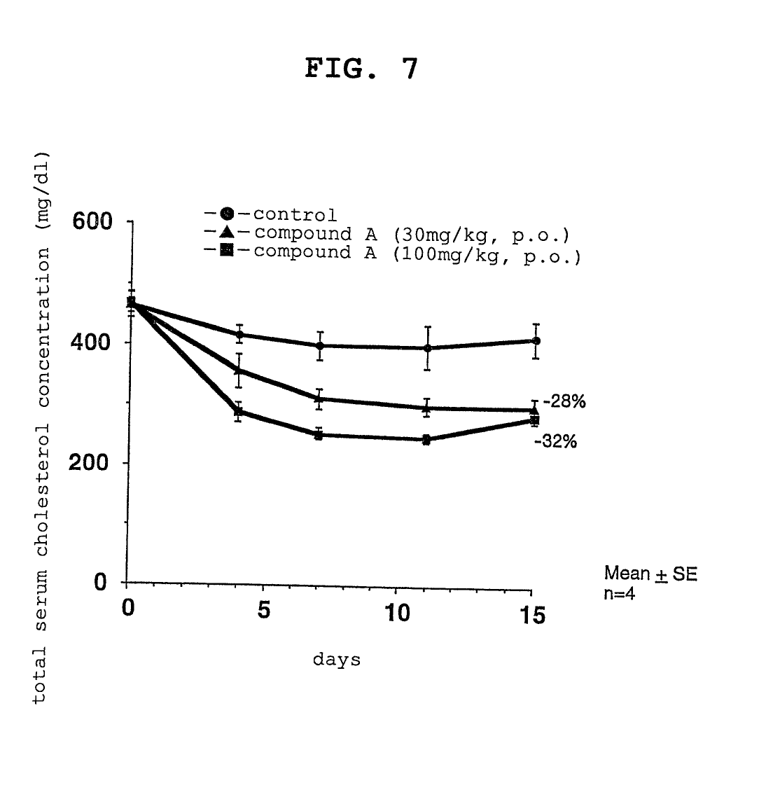

The results are shown in FIG. 6. In the forcible single oral administration of compound A (100 mg/kg), Tₘₐₓ was 6 hours and Cₘₐₓ was 2010 ng/ml.

### Example 9

Effect of Compound A on Serum Cholesterol, Hepatic Expression Amount of CYP7A Gene and Ileal Expression Amount of I-BABP Gene

The total serum cholesterol-lowering action of compound A was examined using hamsters.

Male Syrian Golden Hamsters (7 weeks old) were purchased from Japan SLC and preliminarily reared for 2 weeks under a high fat diet load (10% coconut oil, 0.12% cholesterol). After the preliminary rearing, the high fat diet was fed to the hamsters while administering compound A. The drug was prepared into a 0.5% methylcellulose suspension, and forcibly administered orally at 30 or 100 mg/kg/day for 14 consecutive days. At 4, 7, 11 and 14 days from the drug administration, blood was drawn from the orbital vein of the animal and total serum cholesterol value was measured. As a control, a drug non-administration group was prepared and treated similarly, which was followed by measurement of the serum total cholesterol value. At the final day of the test, the liver and the ileum were removed from the animals, from which the total RNA was prepared.

(1) Serum Total Cholesterol Value

The blood was drawn from the orbital vein of the animals and centrifuged (3000 rpm, 15 min) to prepare the serum. The total cholesterol concentration of the serum was determined by the enzyme method.

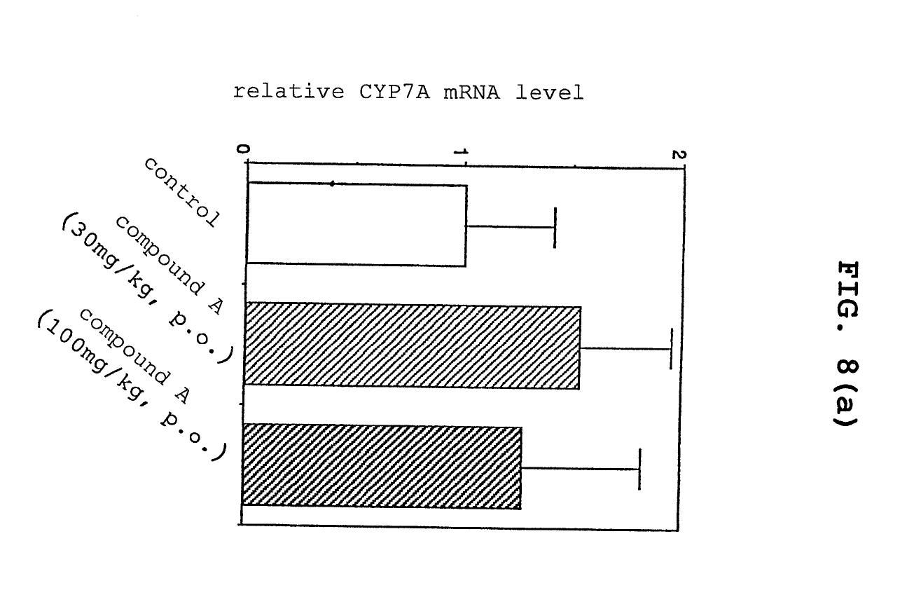

The results are shown in FIG. 7. The serum total cholesterol value decreased by the administration of compound A. The percent decrease in serum total cholesterol on the last day of the test of the compound A (30 and 100 mg/kg) administration group relative to the drug non-administration group was 28% and 32%, respectively.

(2) Northern Blot Analysis for CYP7A Gene and I-BABP Gene

The influence of the administration of compound A on the expression of CYP7A gene and I-BABP gene in hamster was examined by the Northern blot method.

The total RNA was prepared from animal tissues by AGPC method. The prepared total RNA was electrophoresed on an agarose gel containing 6% formaldehyde and transferred onto a nylon membrane by a capillary blotting method. This membrane was hybridized with ³²P-labeled hamster CYP7A or I-BABP probe in 50% formamide at 42° C. for 16 h and the amounts of CYP7A and I-BABP gene expressions were measured. The CYP7A and I-BABP probes were prepared by RT-PCR method from RNA derived from the liver and the ileum, respectively, of the hamsters.

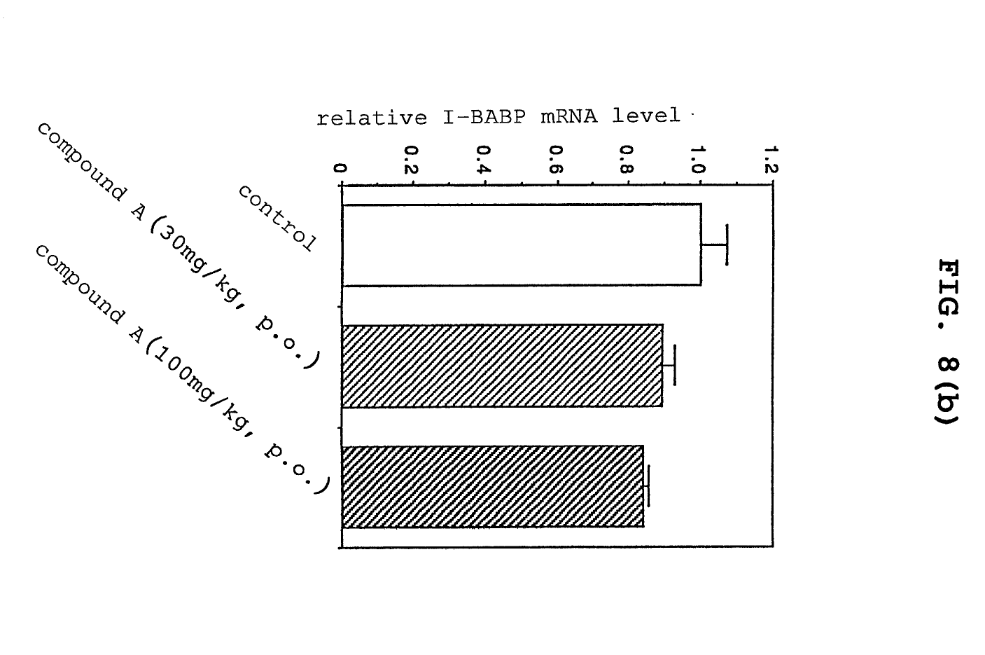

The results are shown in FIG. 8. In the liver, the mRNA level of the CYP7A gene increased in the compound A administration group (53% and 27% increase in 30 and 100 mg/kg administration groups, respectively, relative to the drug non-administration group, FIG. 8A). The mRNA level of the I-BABP gene in the ileum decreased in the compound A administration group (11% and 16% decrease in the 30 and 100 mg/kg administration groups, respectively, relative to the drug non-administration group, FIG. 8B).

Based on the above results, it is considered that compound A antagonized the transcription activity-promoting action of FXR, caused an increase in the expression of CYP7A gene in the liver and a decrease in the I-BABP gene expression in the ileum, whereby the serum total cholesterol-lowering action was exhibited.

The FXR antagonist obtained according to the screening method of the present invention causes an increased expression of CYP7A gene in the liver and a decreased I-BABP gene expression in the ileum, and shows a serum total cholesterol-lowering action. Therefore, it is useful for the prophylaxis and treatment of the diseases relating to the cholesterol metabolism and the transport of bile acid, particularly for the prophylaxis and treatment of hyperlipidemia wherein the main symptom is increase in serum cholesterol.

### SEQUENCE LISTING FREE TEXT

SEQ ID NO:1 “Xaa” means any amino acid.

SEQ ID NO:2 Oligonucleotide designed to act as RT-PCR primer.

SEQ ID NO:3 Oligonucleotide designed to act as RT-PCR primer.

SEQ ID NO:4 Oligonucleotide designed to act as RT-PCR primer.

What is claimed is:

1. A method for treating hyperlipidemia, which comprises administering a pharmaceutically effective amount of a farnesoid X receptor antagonist to a patient.

2. The method of claim 1, wherein the farnesoid X receptor antagonist has an IC₅₀ value of not more than 10 μM.

3. The method of claim 1, wherein the IC₅₀ value of the farnesoid X receptor antagonist is not more than 10 μM when the ligand is 100 μM.

4. The method according to claim 3, wherein the farnesoid X receptor ligand is a bile acid.

5. The method according to claim 4, wherein the bile acid is chenodeoxycholic acid, deoxycholic acid, lithocholic acid, ursodeoxycholic acid or 3,7-diketocholanic acid.

6. The method according to claim 1, wherein the farnesoid X receptor antagonist is N-(3,5-di-tert-butyl-2,6-dihydroxyphenyl)benzamide or a pharmaceutically acceptable salt thereof.

7. A method for treating hyperlipidemia, which comprises repressing a ligand dependent action of farnesoid X receptor.

8. A method for promoting biosynthesis of bile acid, which comprises increasing an expression of a cholesterol 7α-hydroxylase (CYP7A) gene or protein.

9. A method for inhibiting re-absorption of bile acid, which comprises repressing an expression of an intestinal bile acid-binding protein (I-BABP) gene or protein.

10. A method for promoting bile acid secretion, which comprises prohibiting decrease of an expression of a bile salt export pump (Bsep) gene or protein.

11. The method according to claim 1 or 7, which shows at least one of the following features (a) to (c):
(a) increase in an expression of a cholesterol 7α-hydroxylase (CYP7A) gene or protein
(b) repression of an expression of an ileum bile acid binding protein (I-BABP) gene or protein
(c) prohibition of decrease in an expression of a bile acid export pump (Bsep) gene or protein.

12. A method for screening a farnesoid X receptor antagonist, which comprises the following steps:
(1) forming, in the presence of bile acid, a complex of farnesoid X receptor or its operable fragment labeled with a first fluorescent dye and a farnesoid X receptor coactivator labeled with a second fluorescent dye,
(2) adding a test compound and incubating the compound, and
(3) measuring an amount of a free coactivator by a fluorescence resonance energy transfer assay method.

13. The screening method according to claim 12, wherein the bile acid is chenodeoxycholic acid.

14. The screening method according to claim 12, wherein the farnesoid X receptor coactivator is selected from the SRC-1 family or an operable fragment of the selected coactivator.

15. A farnesoid X receptor antagonist obtainable by the screening method of any of claim 12 to claim 14.

16. A method for treating hyperlipidemia, which comprises administering a pharmaceutically effective amount of a farnesoid X receptor antagonist to a patient.
